(19) United States
(12) Patent Application Publication
(10) Pub. No.: US 2012/0238915 A1
(43) Pub. Date: Sep. 20, 2012

(54) SOUND WAVE TREATMENT PLATFORM AND SOUND WAVE TREATMENT METHOD

(76) Inventor: Der-Yang TIEN, Taipei (TW)

(21) Appl. No.: 13/420,971

(22) Filed: Mar. 15, 2012

Related U.S. Application Data
(60) Provisional application No. 61/453,320, filed on Mar. 16, 2011.

(51) Int. Cl. A61N 7/00

(52) U.S. Cl. 601/2

(57) ABSTRACT

A sound wave treatment platform is provided. The sound wave treatment platform includes a sound wave emitting module, a control module and a supporting component for receiving and supporting the sound wave emitting module. The sound wave emitting module includes a plurality of sound wave emitting devices for emitting non-focused sound waves. A sound wave treatment method is also provided. The sound wave treatment method includes the step of administering non-focused sound waves to a subject, wherein the non-focused sound waves are produced by a plurality of sound wave emitting devices.

### BACKGROUND OF THE INVENTION

1. Field of the Invention

The present invention relates to a treatment platform, and more particularly, to a sound wave treatment platform.

2. Description of Related Art

In Taiwan, cancers are the first cause of deaths in past 28 years. According to the statistics from WHO, the total number of deaths resulting from cancers is 7.9 millions in 2007, and it predicts that the number of deaths resulting from cancers would be 12 millions in 2030. Currently, chemical treatments are the main stream for treating cancers. However, the delivery of an anti-cancer drug to a tumor tissue is not easily controlled due to resistance to the anticancer drug and high tumor interstitial fluid pressure.

In other words, the interstitial fluid pressure (IFP) of a tumor tissue is higher than that of a normal tissue due to tumor vessel leakiness, lack of lymph vessels, fibrosis of the tumor tissue and fibrosis of mesenchymal cells. Further, the high interstitial fluid pressure (IFP) of a tumor tissue forms the barrier of the tumor tissue that prevents small molecular drugs and antibodies from being delivered into the tumor tissue. Therefore, only a small portion of the drugs may be delivered into the tumor tissue, and most of the drugs are distributed in blood, thereby adversely affecting the treatment and producing many side effects. In order to avoid side effects of anti-tumor drugs, the lower concentration of drags is administered, and thus cannot kill tumor cells completely. Many methods have been developed to significantly accumulate anti-tumor drugs at tumor tissues, such that tumor cells may expose to the locally high concentration of the anti-tumor drug. Accordingly, the tumor cells can be effectively killed, and the side effects are also reduced.

Liposomes were developed by Alec Bangham in 1965. Liposomes have great biocompatibility and biodegradability, and form spherical micelles in aqueous environment. Therefore, liposomes are widely used in the drug delivery. In addition, liposomes may extend the half-life of drugs in the circulating system, such that the drugs may be taken by tumor cells. In the recent years, a lot of anti-tumor drugs carried by liposomes have thus been developed. Further, tumor cells grow rapidly, and secrete more vascular permeability factors. The space (about 100-800 nm) among endothelial cells in a tumor tissue is larger than the space (about 5-10 nm) among endothelial cells in a normal tissue, such that macromolecules (such as liposomes) may be delivered into the tumor tissue rather than the normal tissue. Further, the tumor tissue has no lymph, such that the liposomes may stay in the tumor tissue, i.e., enhanced permeability and retention effect (EPR effect). In comparison with small molecular drugs, the drug concentration in the tumor tissue may be increased by passive targeting of the anti-tumor drug carried by the liposomes, so as to enhance the efficacy of the treatment and to decrease the side effects of the drug.

However, the tumor tissue grows rapidly and has no contact inhibition, such that the cell density of the tumor tissue is significantly higher than that of the normal tissue. Hence, it is an urgent issue in the tumor treatment to enhance bioavailability of a drug in a tumor tissue, so as to improve efficacy of the treatment.

### SUMMARY OF THE INVENTION

The present invention provides a sound wave treatment platform, which includes a sound wave emitting module, a control module and a supporting component. The sound wave emitting module includes a plurality of sound wave emitting devices for emitting non-focused sound waves. The control module is connected to the sound wave emitting module for controlling the sound wave emitting module. The supporting component is configured to receive and support the sound wave emitting module. The sound wave emitting devices are used at a frequency no more than 0.5 MHz. According to an embodiment of the present invention, the sound wave emitting devices are low-frequency ultrasonic transducers. According to an embodiment of the present invention, the sound wave emitting devices are tweeters. According to an embodiment of the present invention, each of the sound wave emitting devices is independently operated by the control module.

According to an embodiment of the present invention, the sound wave treatment platform further includes a connecting component for connecting the supporting component and the control module. According to an embodiment of the present invention, the supporting component is a bed. According to an embodiment of the present invention, the supporting component has an elastic element for supporting the sound wave emitting devices of the sound wave emitting module. According to an embodiment of the present invention, the non-focused sound waves are conducted to a subject to be treated via a gel pad.

The sound wave treatment platform of the present invention is used for a systemic treatment or a local treatment.

According to an embodiment of the present invention, the sound wave treatment platform is used with a drug treatment for enhancing the efficacy of the drug treatment. According to an embodiment of the present invention, the sound wave treatment platform is used with an anti-tumor drug treatment for enhancing efficacy of the anti-tumor drug treatment. The anti-tumor drug treatment includes a pharmaceutical composition having a tumor targeting characteristic, a pharmaceutical composition without a tumor targeting characteristic or a combination thereof. According to an embodiment of the present invention, the anti-tumor treatment includes a nano-carrier composition having nanoparticles. According to an embodiment of the present invention, the anti-tumor treatment includes a liposome composition. According to an embodiment of the present invention, the anti-tumor treatment is used for treating a cancer.

The present invention further provides a sound wave treatment method, including the step of administering non-focused sound waves to a subject, wherein the non-focused sound waves are produced by a plurality of sound wave emitting devices, and the sound wave emitting devices are used at a frequency no more than 0.5 MHz. According to an embodiment of the present invention, the sound wave emitting devices are low-frequency ultrasonic transducers, and the non-focused sound waves are non-focused ultrasonic waves. According to an embodiment of the present invention, the sound wave emitting devices are tweeters such as high-frequency tweeters.

According to an embodiment of the present invention, the non-focused sound waves are conducted to the subject via a gel pad, wherein the gel pad is disposed between the sound wave emitting devices and the subject. According to an embodiment of the present invention, the sound wave emitting devices are supported by a supporting component. According to an embodiment of the present invention, the supporting component is a bed. According to an embodiment of the present invention, each of the sound wave emitting devices is independently operated. According to an embodiment of the present invention, the above-mentioned sound wave treatment platform is used for administering non-focused sound waves to the subject.

The sound wave treatment method of the present invention is used for a systemic treatment or a local treatment.

According to an embodiment of the present invention, the sound wave treatment method further includes the step of administering a drug to the subject, wherein the non-focused sound waves enhance efficacy of the drug treatment. According to an embodiment of the present invention, the drug is an anti-tumor drug, and the efficacy of the anti-tumor treatment is enhanced by the non-focused sound waves. The anti-tumor drug may be a pharmaceutical composition having a tumor targeting characteristic, a pharmaceutical composition without a tumor targeting characteristic or a combination thereof. According to an embodiment of the present invention, the anti-tumor drug is a nano-carrier composition having nanoparticles. According to an embodiment of the present invention, the antitumor drug is a liposome composition. According to an embodiment of the present invention, the anti-tumor drug is an anti-cancer drug.

### BRIEF DESCRIPTION OF THE DRAWINGS

FIG. 1 is a schematic view showing the sound wave treatment platform of the present invention;

FIG. 2 is a schematic view showing the operation of the sound wave treatment platform according to an embodiment of the present invention;

FIG. 3 is a schematic view showing an embodiment of the present invention;

FIG. 4 is a schematic view showing an embodiment of the present invention;

FIG. 5A and FIG. 5B are schematic views showing an embodiment of the present invention;

FIG. 6 is a schematic view showing an embodiment of the present invention;

FIG. 7 is a schematic view showing an embodiment of the present invention;

FIG. 8 is a schematic view showing an embodiment of the present invention; and

FIG. 9 is a schematic view showing an embodiment of the present invention.

### DETAILED DESCRIPTION OF THE PREFERRED EMBODIMENTS

The detailed description of the present invention is illustrated by the following specific examples. Persons skilled in the art can conceive the other advantages and effects of the present invention based on the disclosure contained in the specification of the present invention.

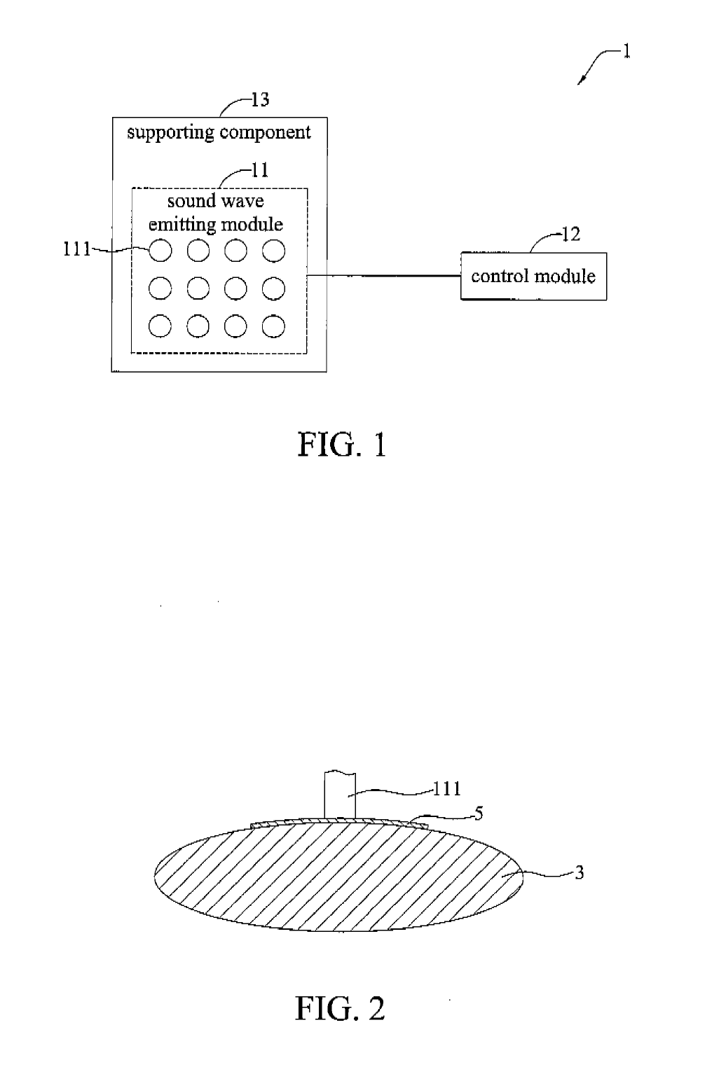

FIG. 1 is a schematic view showing a sound wave treatment platform of the present invention. As shown in FIG. 1, the sound wave treatment platform 1 includes a sound wave emitting module 11, a control module 12 and a supporting component 13. The supporting component 13 is used for receiving and supporting the sound wave emitting module 11. The sound wave emitting module 11 has a plurality of sound wave emitting devices 111. The control module 12 is connected for controlling the sound wave emitting module 11. Each of the sound wave emitting devices 111 in independently controlled and operated by the control module 12. The sound wave treatment platform 1 produces sound waves by the sound wave emitting devices 111 for a systemic treatment or a local treatment, wherein the systemic treatment includes more than one local treatment. In addition, each of the sound wave emitting devices 111 may be independently operated, and may be optionally used according to various treatment conditions (such as portions to be treated, areas to be treated or conditions of diseases). In other words, the sound wave emitting device may be specifically and optionally activated.

The sound wave emitting module 11 produces non-focused sound waves by the sound wave emitting devices 111. There is no limitation to the type of the sound wave emitting devices 111. Any type of sound wave emitting devices for producing non-focused sound waves may be used in the present invention. The sound wave emitting devices 111 may be low-frequency ultrasonic transducers. The sound wave emitting module 11 produces non-focused ultrasonic waves by a plurality of low-frequency ultrasonic transducers. The sound wave emitting devices 111 may be tweeters such as high-frequency tweeters.

In one embodiment, low-frequency ultrasonic transducers may be the sound wave emitting devices 11. The conventional ultrasonic treatment technology uses only one transducer for the treatment, such that the treatment takes longer time for treating larger areas. The sound wave treatment platform 1 of the present invention produces sound waves by using a plurality of sound wave emitting devices 111, so as to provide convenient treatment. In addition, in the conventional ultrasonic treatment, the ultrasonic transducer has to move or rotate; in other words, there is a corresponding movement between the transducer and the subject, so as to avoid thermal injury to the tissue of the subject. Optionally, the sound wave emitting devices 11 of the sound wave treatment platform 1 may fixedly contact the subject during the treatment, and no thermal injury occurs in the tissue of the subject. Thus, in comparison with the prior art, the sound wave treatment platform of the present invention is safer and more convenient. In the present invention, “fixedly contact” means that the sound wave emitting devices 111 have no movement relatively to the subject during the treatment by the sound wave emitting devices 111. In the treatment, the sound wave treatment platform 1 produces non-focused sound waves for more than 10 minutes, preferably more than 15 minutes, and more preferably more than 25 minutes. According to an embodiment of the present invention, the sound wave emitting devices 111 of the sound wave treatment platform 1 fixedly contact the subject in the treatment for more than 10 minutes, preferably more than 15 minutes, and more preferably more than 25 minutes.

The sound wave emitting devices 111 are used at a frequency no more than 0.5 MHz, preferably no more than 0.4 MHz, and more preferably no more than 0.3 MHz. Further, the sound wave emitting devices 111 are used at the strength no more than 5 W/cm², preferably no more than 4 W/cm², and more preferably no more than 3 W/cm².

The control module 12 is electrically connected to the sound wave emitting module 11 for controlling the sound wave emitting module 11. Each of the sound wave emitting devices 111 of the sound wave emitting module is independently operated by the control module 12. The sound wave emitting devices 111 are activated by the control module. The number of the activated sound wave emitting devices 111 is set by the control module 12. The sound wave emitting devices 111 are selectively activated by the control module 12 according to the portion to be treated. Further, parameters such as strength, frequency, waveform, duration and contact angles of the sound wave emitting devices 111 may be set by the control module 12. A conventional electrical connecting component may be used for connecting the control module 12 and the sound wave emitting module 11. In addition, the control module 12 may be connected to the supporting component 13.

In the present invention, the waveforms may be, but not limited to, sine waves, triangle waves or square waves. Further, the sound waves produced by the sound wave treatment platform 1 may be continuous waves or pulsed waves. According to an embodiment of the present invention, the sound emitting module 11 of the sound wave treatment platform 1 produces continuous waves by a plurality of sound wave emitting devices 111. The subject may be treated by the continuous waves emitted from the sound wave treatment platform 1 without thermal injure to tissues of the subject. The sound wave treatment platform 1 produces non-focused sound waves, which are continuous waves, to treat the subject for more than 10 minutes, preferably more than 15 minutes, and more preferably more than 25 minutes.

The medium such as water, a gel or a gel pad may be used for enhancing the conduction of the sound waves in the present invention. FIG. 2 shows the operation of the present invention. While using the sound wave treatment platform 1 of the present invention, the medium such as a gel pad 5 may be disposed between the sound wave emitting devices 111 of the sound wave emitting module 11 and the portions of the subject 3 to be treated, so as to enhance the conduction efficiency of the sound waves. The shape, the size and the amount of the gel pad 5 are not limited, and may be optionally selected according to the requirements of the treatment. The gel pads may be respectively disposed on each of the sound wave emitting devices 111, or a single gel pad may be used to cover all the sound wave emitting devices 111 of the sound wave emitting module 11. Alternatively, the gel pad may cover some sound wave emitting devices 111 of the sound wave emitting module 11 according to the portion of the subject 3 to be treated.

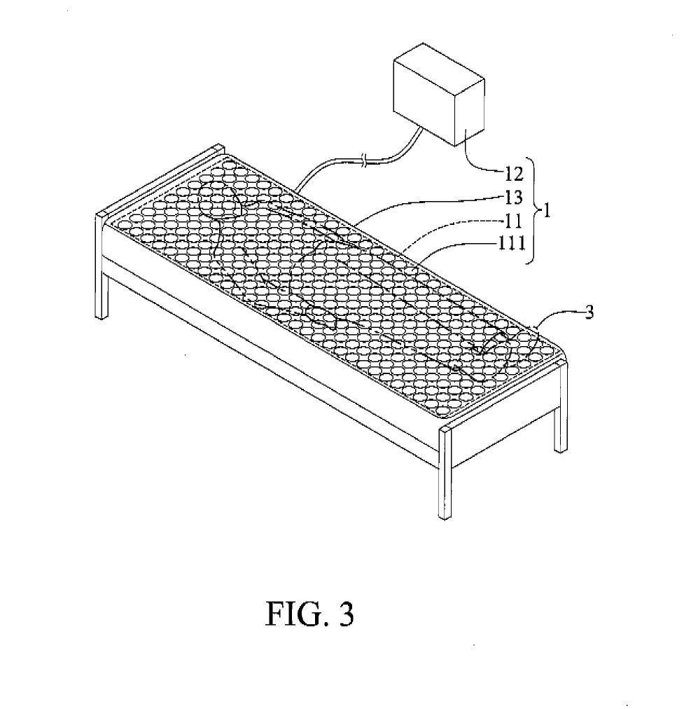

There is no limitation to the type of the supporting component 13 for receiving and supporting the sound wave emitting module 11. FIG. 3 shows an embodiment of the present invention. As shown in FIG. 3, the supporting component 13 may be a bed for receiving and supporting the sound wave emitting module 11. The subject 3 to be treated may be at any posture such as lying, lying on the side, lying face downward, sitting or standing. The sound wave treatment platform 1 may be used for a systemic treatment or a local treatment to the subject via the design of the supporting component 13. The systemic treatment includes many local treatments on the subject. The control module 12 is electrically connected to the sound wave emitting module 11. Each of the sound wave emitting devices 111 of the sound wave emitting module 11 may be independently operated by the control module 12. For example, the sound wave emitting devices may be low-frequency ultrasonic transducers, and the sound wave emitting module 11 may produce non-focused ultrasonic waves by the low-frequency ultrasonic transducers. The sound wave emitting devices 111 may be tweeters. The specific ones of the sound wave emitting devices 111 may be activated by the control module 12 according to the portion to be treated. The parameters such as strength, frequency, waveforms, duration and contact angles of the sound wave emitting devices 111 may be set by the control module 12 according to the requirements of the treatment. Optionally, the sound wave emitting devices 111 may fixedly contact the portion of the subject 3 to be treated, and a medium such as a gel pad may be disposed between the sound wave emitting devices 111 of the sound wave emitting module 11 and the portion of the subject 3 for enhancing the conduction efficiency. Further, the control module 12 may be connected to the supporting component 13 for adjusting the state of the supporting component 13 such as the height, tilt or position.

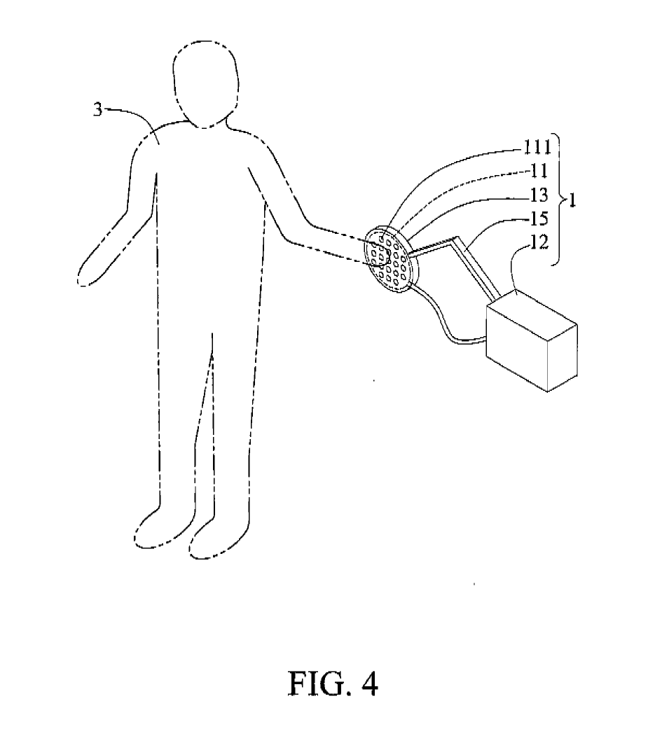

FIG. 4 shows an embodiment of the present invention. As shown in FIG. 4, the supporting component 13 may be formed as a plate with any shape such as a circle, a rectangle, a triangle or a polygon. Any type of the supporting component 13 may be used for receiving and supporting the sound wave emitting module 11 in the present invention. The subject 3 to be treated may be at any posture such as lying, lying on the side, lying face downward, sitting or standing. The sound wave treatment platform 1 may be used for a systemic treatment or a local treatment to the subject via the design of the supporting component 13. The systemic treatment includes many local treatments on the subject. The control module 12 is electrically connected to the sound wave emitting module 11. Each of the sound wave emitting devices 111 of the sound wave emitting module 11 may be independently operated by the control module 12. For example, the sound wave emitting devices may be low-frequency ultrasonic transducers, and the sound wave emitting module 11 may produce non-focused ultrasonic waves by the low-frequency ultrasonic transducers. The sound wave emitting devices 111 may be tweeters. The specific ones of the sound wave emitting devices 111 may be activated by the control module 12 according to the portion to be treated. The parameters such as strength, frequency, waveforms, duration and contact angles of the sound wave emitting devices 111 may be set by the control module 12 according to the requirements of the treatment. Optionally, the sound wave emitting devices 111 may fixedly contact the portion of the subject 3 to be treated, and a medium such as a gel pad may be disposed between the sound wave emitting devices 111 of the sound wave emitting module 11 and the portion of the subject 3 for enhancing the conduction efficiency. Further, the control module 12 may be connected to the supporting component 13. As shown in the figure, the control module 12 may be connected to the supporting component 13 via a connecting component. The connecting component 15 may be a fix type or a movable type for adjusting the state of the supporting component 13 such as the height, tilt or position. The position of the supporting component 13 may be set by the control module 12 via the connecting component 15 for adjusting the position of the sound wave emitting module received in the supporting component 13 relative to the subject 3. There is no limitation to the type, the size and the material of the connecting component 15 and to the connection of the connecting component 15, the supporting component 13 and the control module 12.

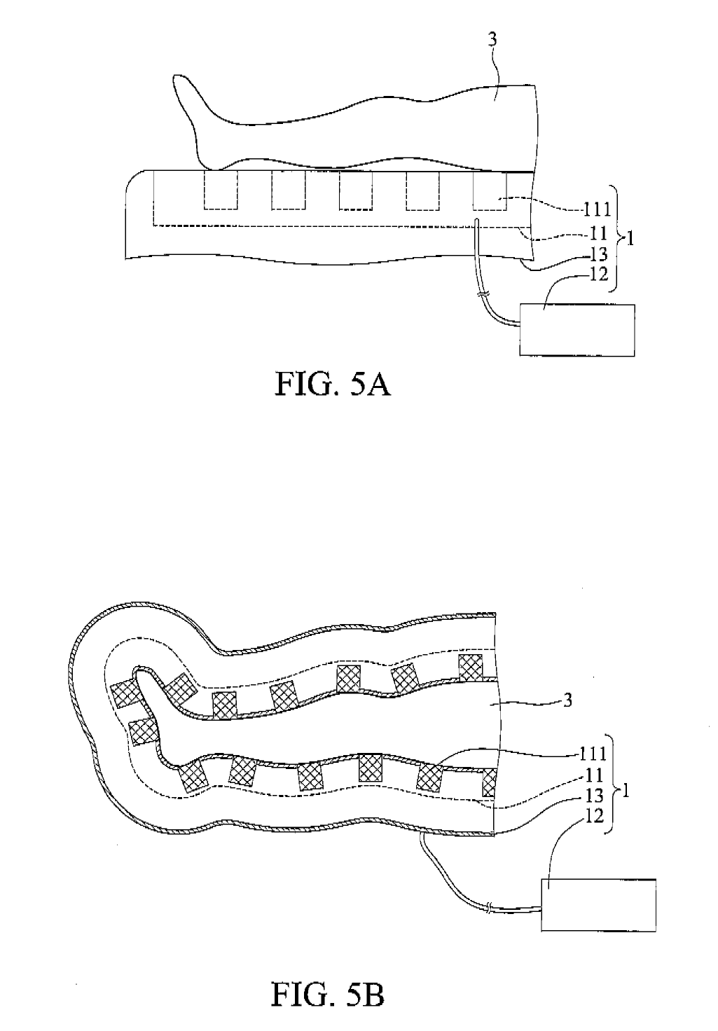

FIG. 5A and FIG. 5B show an embodiment of the present invention. The supporting component 13 may be formed as an air bag made of any material and having any shape for receiving the sound wave emitting module 11. The subject 3 to be treated may be at any posture such as lying, lying on the side, lying face downward, sitting or standing. For example, as shown in FIG. 5A, the subject 3 was lying in contact with the supporting component 13. As shown in FIG. 5B, the supporting component 13 may be any type such as a magic tape, a tying tape or a zip. The sound wave treatment platform 1 may be used for a systemic treatment or a local treatment to the subject 3 via the design of the supporting component 13. The systemic treatment includes many local treatments on the subject. The control module 12 is electrically connected to the sound wave emitting module 11. Each of the sound wave emitting devices 111 of the sound wave emitting module 11 may be independently operated by the control module 12. For example, the sound wave emitting devices 111 may be low-frequency ultrasonic transducers, and the sound wave emitting module 11 may produce non-focused ultrasonic waves by the low-frequency ultrasonic transducers. The sound wave emitting devices 111 may be tweeters. The specific ones of the sound wave emitting devices 111 may be activated by the control module 12 according to the portion to be treated. The parameters such as strength, frequency, waveforms, duration and contact angles of the sound wave emitting devices 111 may be set by the control module 12 according to the requirements of the treatment. Optionally, the sound wave emitting devices 111 may fixedly contact the portion of the subject 3 to be treated, and a medium such as a gel pad may be disposed between the sound wave emitting devices 111 of the sound wave emitting module 11 and the portion of the subject 3 for enhancing the conduction efficiency. Further, the control module 12 may be connected to the supporting component 13 for adjusting the state of the supporting component 13 such as the relative position to the subject 3 or gas-filling state.

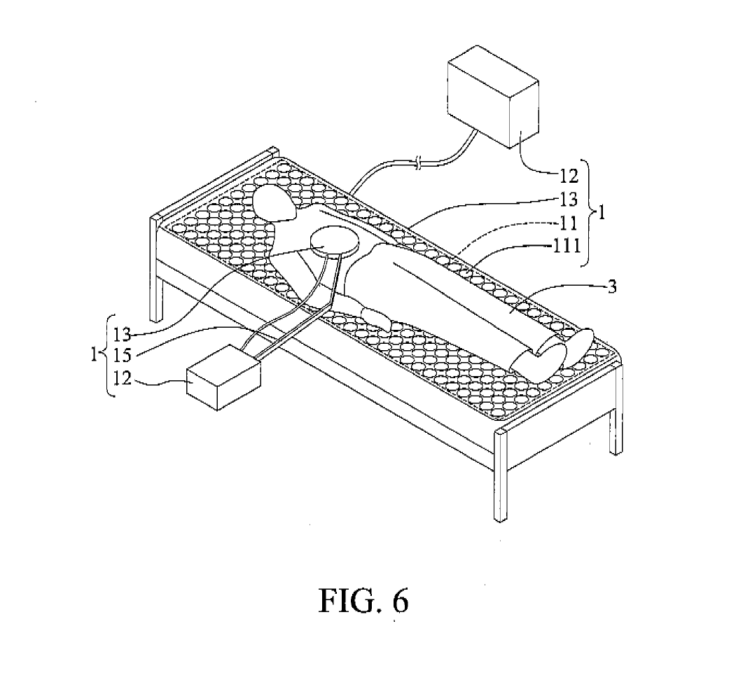

In addition, the sound wave treatment platform has at least a supporting component to meet various requirements of the treatment and to enhance the efficacy of the treatment. Various types of supporting components may be used. For example, the supporting components in the previous embodiments may be used in combination. Certainly, more than one sound wave treatment platform may be used for the treatment. FIG. 6 shows an embodiment of the present invention. As shown in FIG. 6, more than one sound wave treatment platform may be used at the same time or separately.

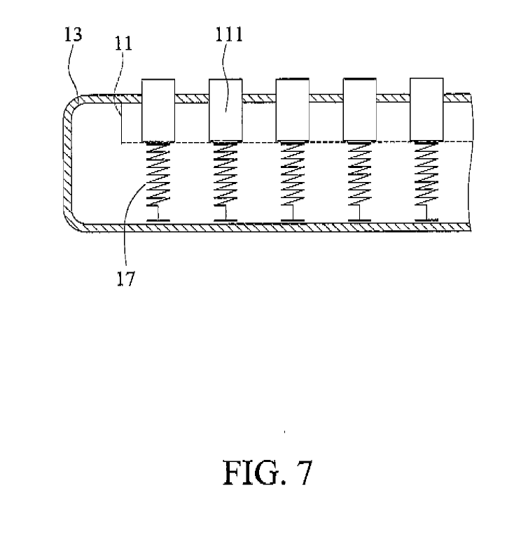

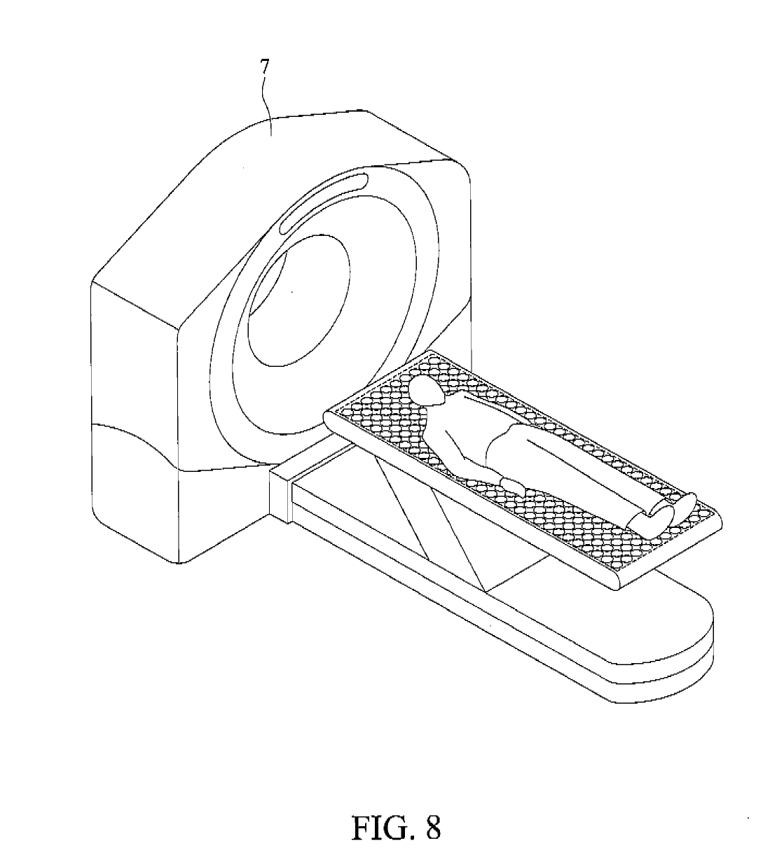

FIG. 7 shows an embodiment of the present invention. The supporting component 13 is used for supporting the sound wave emitting devices 111 of the sound wave emitting module 11 via an elastic element 17 such as a coil spring or a spring pad so as to facilitate the contact of the sound wave emitting devices 111 of the sound wave emitting module 11 and the portion to be treated, and to enhance the conduction efficiency. One or more elastic elements may be used for supporting the sound wave emitting devices of the sound wave emitting module. There is no limitation to the connection of the elastic element 17 and the sound wave emitting module 11 or the sound wave emitting devices 111.

The sound wave treatment platform 1 of the present invention may use a plurality of sound wave emitting devices 111 for producing sound waves for a systemic treatment or a local treatment. The systemic treatment includes many local treatments. For example, the sound wave treatment platform 1 is used for treating a disease such as a pain, a lung disease, an inflammation, an infection, diabetes or obesity.

The sound wave treatment platform of the present invention may be used with a drug treatment so as to enhance the efficacy of the drug treatment. The drug may be, but not limited to, the drug used for treating the previous diseases. The sound wave treatment platform of the present invention produces non-focused sound waves at a frequency no more than 0.5 MHz to enhance drug bioavailability.

The sound wave treatment platform of the present invention may be used for treating tumors, wherein the treatment may be a systemic treatment or a local treatment. For example, the sound wave treatment platform may be used for treating malignant tumors. The malignant tumors may translocate to other tissues via the blood circulation or lymph system, so as to result in multiple systemic metastasis. It is not easy to detect the early stage of the metastasis, such that the treatment is very difficult. Thus, metastases are the major cause of death from cancers. The sound wave treatment platform 1 of the present invention may produce sound waves by a plurality of sound wave emitting devices 111 to perform a systemic treatment. Hence, the sound wave treatment platform of the present invention may enhance the cancer treatment.

The sound wave treatment platform of the present invention may be used with the conventional anti-tumor treatment. For example, the sound wave treatment platform of the present invention may be used with the anti-tumor treatment to enhance the efficacy of the anti-tumor treatment. According to an embodiment of the present invention, an anti-cancer drug may be used. The sound wave treatment platform of the present invention may enhance the efficacy of the cancer treatment, eliminate pain, reduce the dosage of the anti-cancer drug and reduce side effects (such as sick, vomiting, diarrhea, hair loss, decrease of leukocytes or inappetence).

The anti-tumor treatment includes a pharmaceutical composition having a tumor targeting characteristic, a pharmaceutical composition without a tumor targeting characteristic or a combination thereof. The nano-carrier composition such as a micelle composition or a nanoparticle composition may be used for the anti-tumor treatment.

The sound wave treatment platform of the present invention has significant efficacy of the anti-tumor treatment. The evaluation items of the tumor treatment efficacy include symptoms, tumor sizes and/or tumor markers. The symptom may be pain. The tumor size is evaluated based on the measurable tumors by the unidimensional measurement (maximal diameter of the tumor) and/or the bidimensional measurement (maximal diameter multiplied by the crossed maximal diameter). The tumor markers may be, but not limited to, α-fetaprotein (AFP), carcinoembryonic antigin (CEA), CA15-3, CA19-9, CA125, prostate specific antigen (PSA), squamous cell carcinoma antigen (SCC) and β-human chorionic gonadotropin (β-HCG).

While treating tumors, the sound wave treatment platform of the present invention alleviates the symptoms, decreases the tumor size and reduces the tumor markers. Hence, the sound wave treatment platform of the present invention provides significant efficacy of the treatment.

Further, in comparison with using the anti-tumor drug alone in the treatment, the sound wave treatment platform may be used with the anti-tumor drug for significantly improving the efficacy of the treatment.

In addition, while treating the late-stage cancer, the sound wave treatment platform of the present invention significantly enhances the efficacy of the treatment. Moreover, for treating the subject having the drug-resistance, the sound wave treatment platform of the present invention also significantly enhances the efficacy of the treatment.

The sound wave treatment platform of the present invention may be used with the conventional detecting device or detecting technology. FIG. 8 shows an embodiment of the present invention. As shown in FIG. 8, the sound wave treatment platform of the present invention may be used with the detecting device 7. The conventional detecting device or technology may be used to identify the portion to be treated and to evaluate the effect of the treatment. The detecting device or technology may be, but not limited to, MRI, CT, SPECT, PET, ultrasonic examinations, x-ray examinations, angiography or a combination thereof.

The present invention also provides a sound wave treatment method. The sound wave treatment method of the present invention includes the step of administering non-focused sound waves to a subject, wherein the non-focused sound waves are produced by a plurality of sound wave emitting devices, and the sound wave emitting devices are used at a frequency no more than 0.5 MHz. For example, the sound wave emitting devices may be low-frequency ultrasonic transducers for producing non-focused ultrasonic waves. The sound wave emitting devices may be tweeters. In the present invention, the sound waves may have any waveforms such as sine waves, triangle waves, square waves and etc. In the present invention, the non-focused sound waves may be continuous waves or pulsed waves. According to an embodiment of the present invention, the non-focused sound waves produced by the sound wave emitting devices are continuous waves. The continuous waves may be used for treating the subject without causing thermal injury. According to the sound wave treatment method of the present invention, the continuous waves may be used to treat the subject for more than 10 minutes, preferably for more than 15 minutes, and more preferably for more than 25 minutes.

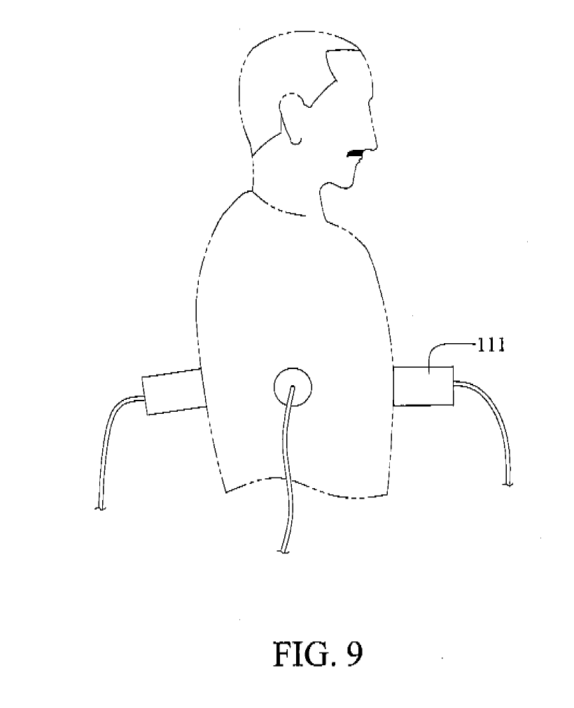

According to the present invention, each of the sound wave emitting devices is independent. FIG. 9 shows an embodiment of the present invention. As shown in FIG. 9, each of the sound wave emitting devices is independent for producing non-focused sound waves.

Further, the plurality of sound wave emitting devices may be supported by the supporting component (for example, shown in FIG. 3 to FIG. 7). In the present invention, the supporting component may be of any type such as a bed, a plate or a combination thereof. According to an embodiment of the present invention, each of the sound wave emitting devices may be independently operated.

In the sound wave treatment method, the medium such as water, a gel or a gel pad may be used to enhance the conduction efficiency. According to an embodiment of the present invention, the non-focused sound waves are conducted to the subject via a gel pad, wherein the gel pad is disposed between the sound wave emitting devices and the subject. Each of the sound wave emitting devices may have a gel pad disposed thereon, or the multiple sound wave emitting devices may be covered by a single gel pad. For example, the gel pads may be disposed for covering multiple sound wave emitting devices according to the portion of the subject to be treated.

According to the sound wave treatment method of the present invention, the sound wave emitting devices may fixedly contact the subject without causing thermal injury in the treatment. Therefore, in comparison with the prior art, the sound wave treatment method of the present invention is safer and more convenient. In the specification of the present invention, “fixedly contact” means that the sound wave emitting devices have no movement relatively to the subject during the treatment. In the treatment method of the present invention, non-focused sound waves are continuously used for more than 10 minutes, preferably more than 15 minutes, and more preferably more than 25 minutes. According to an embodiment of the present invention, the sound wave emitting devices may fixedly contact the subject in the treatment for more than 10 minutes, preferably more than 15 minutes, and more preferably more than 25 minutes.

In the sound wave treatment method of the present invention, the non-focused sound waves are produced by a plurality of sound wave emitting devices, and the sound wave emitting devices are used at a frequency no more than 0.5 MHz, preferably no more than 0.4 MHz, and more preferably no more than 0.3 MHz. Further, the sound wave emitting devices are used at the strength no more than 5 W/cm², preferably no more than 4 W/cm², and more preferably no more than 3 W/cm².

According to an embodiment of the present invention, the sound wave treatment platform 1 may be used for administering non-focused sound waves to the subject.

The sound wave treatment method of the present invention may be used for a systemic treatment or a local treatment, wherein the systemic treatment may include many local treatments. Therefore, in comparison with the prior art, the sound wave treatment method of the present invention is safer and more convenient.

For example, the sound wave treatment method of the present invention is used for treating a disease such as a pain, a lung disease, an inflammation, an infection, diabetes or obesity.

In the present invention, the sound wave treatment method further includes the step of administering a drug to the subject, wherein the efficacy of the drag treatment is enhanced by the non-focused sound waves. For example, the drug may be the conventional drug for treating the above-mentioned disease. According to the sound wave treatment method of the present invention, the subject may be administered with non-focused sound waves at a frequency no more than 0.5 MHz, so as to enhance drug bioavailability.

The sound wave treatment method of the present invention may be used for treating tumors. The sound wave treatment method of the present invention may be used for a systemic treatment or a local treatment. For example, the sound wave treatment method of the present invention may be used for treating cancers. The sound wave treatment method of the present invention may be used with the conventional anti-cancer treatment.

According to the sound wave treatment method of the present invention, the anti-tumor drug may be the conventional antitumor drug. According to an embodiment of the present invention, the anti-tumor drug may be an anti-cancer drug. The sound wave treatment method of the present invention enhances the efficacy of the tumor treatment, effectively alleviates pain, reduces the dosage of the anti-tumor drug and decreases side effects (such as sick, vomiting, diarrhea, hair loss, decrease of leukocytes or inappetence).

The anti-tumor treatment includes a pharmaceutical composition having a tumor targeting characteristic, a pharmaceutical composition without a tumor targeting characteristic or a combination thereof. According to an embodiment of the present invention, the anti-tumor drug is a pharmaceutical composition having a tumor targeting characteristic. The pharmaceutical composition having the tumor targeting characteristic may significantly accumulate at the tumor tissue, and the sound wave treatment method of the present invention may administer non-focused sound waves at a frequency no more than 0.5 MHz to the subject for enhancing the bioavailability of the anti-tumor drug.

According to an embodiment of the present invention, the anti-tumor drug is a nano-carrier composition. The nano-carrier may be, but not limited to, liposomes, micelles or nanoparticles. According to an embodiment of the present invention, the anti-tumor drug is a liposome composition. The nano-carrier composition may significantly accumulate at the tumor tissue due to enhanced permeability and retention effect (EPR effect), i.e., passive targeting. In the sound wave treatment method of the present invention, the subject is administered with the non-focused sound wave at a frequency no more than 0.5 MHz, so as to enhance the drug releasing from the nano-carrier. Further, in the sound wave treatment method of the present invention, the non-focused sound waves at a frequency no more than 0.5 MHz are administered to the subject, so as to change the characteristic of the tumor tissue and to enhance the distribution of the drug in the tumor tissue. In the present invention, the nano-carrier composition includes specific tumor targeting ligands or has no specific tumor targeting ligands. In the sound wave treatment method of the present invention, the non-focused sound waves at a frequency no more than 0.5 MHz are administered to the subject for enhancing bioavailability of the drug.

The pharmaceutical composition without the tumor targeting characteristic may be used as the anti-tumor drug. According to an embodiment of the present invention, the subject is administered with a pharmaceutical composition without the tumor targeting characteristic, and administered with the non-focused sound waves for the systemic treatment.

According to an embodiment of the present invention, the subject may be administered with the pharmaceutical composition, which has no tumor targeting characteristic, and administered with the non-focused sound waves for the local treatment. As shown in FIG. 9, the sound wave emitting devices are disposed on different portions of the subject for emitting sound waves, wherein the sound waves cross at the specific portions to be treated, and the non-focused sound waves increase the bioavailability of the drug entered into the portions to be treated, so as to enhance the efficacy of the drug treatment.

The sound wave treatment method of the present invention may be used to administer non-focused sound waves to the subject before, after or along with the anti-tumor drug treatment. The anti-tumor drug may be, but not limited to, alkylating agents, antimetabolites, antitumor antibiotics, angiogenesis inhibitors, biologic response modifiers, antimicrotubule agents, topoisomerase inhibitors, hormone agents, agents for molecular targeted therapy, cytoprotective agents, pharmaceutical acceptable salts thereof, nano-carrier compositions thereof, and the combination thereof.

The sound wave treatment method of the present invention has significant efficacy for treating tumors. The evaluation items of the tumor treatment efficacy include symptoms, tumor sizes and/or tumor markers. The symptom may be pain. The tumor size is evaluated based on the measurable tumors by the unidimensional measurement (maximal diameter of the tumor) and/or the bidimensional measurement (maximal diameter multiplied by the crossed maximal diameter). The tumor markers may be, but not limited to, α-fetaprotein (AFP), carcinoembryonic antigin (CEA), CA15-3, CA19-9, CA125, prostate specific antigen (PSA), squamous cell carcinoma antigen (SCC) and β-human chorionic gonadotropin (β-HCG).

While treating tumors, the sound wave treatment method of the present invention alleviates the symptoms, decreases the tumor size and reduces the tumor markers. Hence, the sound wave treatment method of the present invention provides significant efficacy of the treatment.

The sound wave treatment method of the present invention may be used for decreasing the dosage of the anti-tumor drug and reducing the side effects.

In addition, while treating the late-stage caner, the sound wave treatment method of the present invention significantly enhances the efficacy of the treatment. Moreover, for treating the subject having the drug-resistance, the sound wave treatment method of the present invention also significantly enhances the efficacy of the treatment.

The sound wave treatment method of the present invention may be used with the conventional detecting device or detecting technology. The conventional detecting device or technology may be used to identify the portion to be treated and to evaluate the effect of the treatment. The detecting device or technology may be, but not limited to, MRI, CT, SPECT, PET, ultrasonic examinations, x-ray examinations, angiography or a combination thereof.

### EXAMPLE 1

An 82 years old female was diagnosed with metastatic pancreatic cancer to liver. Treatment history was as follows: Gemcitabine, Tykerb, Xeloda, and 6000R radiation therapy. Despite multiple treatments, patient's condition continues to worsen with increased abdominal pain. The patient was taking pain medication with constipation.

The patient was treated according to the present invention with 30 mg Bleomycin (Frequency: 0.3 MHz; power intensity: less than 2 W/cm²; duration: less than 30 minutes). After one treatment according to the present invention, the patient's pain was dissolved, and no side effect was observed. The patient has not taken any pain medication since the treatment, and her constipation symptom was also relieved after the treatment. Her tumor marker CA19-9 decreased from 1465 to 406 in 9 days after the treatment. She remained pain free for 150 days after the treatment till her death clue to organ failure.

### EXAMPLE 2

A 62 years old female was diagnosed with recurrent pancreatic cancer after Whipple's surgery. She was declared terminal. Treatment history was as follows: Gemcitabine as a chemotherapeutic agent for 6 months after the surgery and Fentanyl 100 mcg patch for 24 hours non stopping severe abdominal pain. Fentanyl patch only reduced the pain. She still needed additional medication for breakthrough pain.

The patient was treated twice (one week apart) according to the present invention with 30 mg Bleomycin each time (Frequency: 0.3 MHz; power intensity: less than 2 W/cm²; duration: less than 30 minutes (each time)). After the first treatment according to the present invention, the patient's pain scale reduced to 0-1, and no side effect was observed. The patient's body weight increased after the treatment, and physical activity returned to near normal. She has not taken any pain medication since the first treatment till her death. She remained pain free for 170 days after the treatment till her death due to cachexia and liver failure. She lived two years after her first diagnosis.

### EXAMPLE 3

A 79 years old female was diagnosed with metastatic pancreatic cancer to liver. Treatment history was as follows: Gemcitabine, Eloxatin, Pemetrexed, 5-FU, Alimta, and 6000R Radiation. She was in severe pain. She had Fetanyl patch and morphine injection.

The patient was treated according to the present invention with 30 mg Bleomycin (Frequency: 0.3 MHz; power intensity: less than 2 W/cm²; duration: less than 30 minutes). After one treatment according to the present invention, the patient's pain totally dissolved without using any pain medication until her death due to sepsis, and no side effect was observed. She was pain free for 32 days.

### EXAMPLE 4

A 53 years old male was diagnosed with metastatic pancreatic cancer to liver and has been treated with Palliative bypass operation. No chemotherapy history. He was in very terminal stage with severe jaundice and great pain. He used Fetanyl patch and additional pain medication. The patient was treated according to the present invention with 30 mg Bleomycin (Frequency: 0.3 MHz; power intensity: less than 2 W/cm²; duration: less than 30 minutes). After one treatment according to the present invention, his pain totally vanished in 6 hours without using anymore pain medication, and no side effect was observed. He remained pain free for 4 days till his death due to hepatic failure.

### EXAMPLE 5

A 62 years old female was first diagnosed with non-operable pancreatic cancer. She complained of abdominal pain. No cancer or chemotherapy history.

The patient was treated twice (one week apart) according to the present invention with 30 mg Bleomycin each time (Frequency: 0.3 MHz; power intensity: less than 2 W/cm²; duration: less than 30 minutes (each time)). One week later, the patient was further treated with 30 mg Bleomycin, followed by 1000 mg Gemcitabine. After the first treatment according to the present invention, her pancreatic region pain resolved, and no side effect was observed. She was an insulin dependent diabetic patient. After the treatment, her blood sugar returned to normal level without using insulin, and remained till her death. After the above treatment, she was further put on weekly Gemcitabine treatment for at least four months. Her tumor only grew 1.4 cm in diameter during this period. She did not have jaundice. She remained pain free for 183 days after the treatment till her death.

The effect of immediate pain symptom relief after the treatment according to the present invention as shown in Examples 1 to 5 is dramatic. Patients returned to clear consciousness after stop using opioids medication. The pain relief effect is long lasting, and patients no longer need strong pain medication after the treatment according to the present invention.

### EXAMPLE 6

A 62 years old female was diagnosed with metastatic breast cancer, and complained of three weeks left shoulder pain.

The patient was treated according to the present invention with 30 mg Bleomycin (Frequency: 0.3 MHz; power intensity: less than 2 W/cm²; duration: less than 30 minutes). After one treatment according to the present invention, no side effect was observed, and she did not have shoulder pain (pain free for over seven months) till her death due to developed brain metastasis.

### EXAMPLE 7

A 50 years old male was diagnosed with metastatic oral cancer to the face, and complained of severe facial pain. He has been treated with multiple surgery and chemoradiation. He presented with a large area of metastatic facial mass, and his right eye was bulging due to tumor infiltration.

The patient was treated according to the present invention with 30 mg Bleomycin (Frequency: 0.3 MHz; power intensity: less than 2 W/cm²; duration: less than 30 minutes). After one treatment according to the present invention, his facial pain totally resolved with no side effect and remained pain free over 33 days till last follow up. The patient's facial tumor decreased in size, and his right eye was less bulging and could rotate.

### EXAMPLE 8

A 39 years old male was diagnosed with terminal hepatoma and liver pain.

The patient was treated according to the present invention with 30 mg Bleomycin (Frequency: 0.3 MHz; power intensity: less than 2 W/cm²; duration: less than 30 minutes). After one treatment according to the present invention, his right quadrant pain vanished with no side effect, and he was pain free till his death 14 days later.

### EXAMPLE 9

A 62 years old female was diagnosed with recurrent breast cancer and metastasis to the liver. She had multiple chemotherapy and target medicine treatment.

The patient was treated twice (one week apart) according to the present invention with 30 mg Bleomycin each time (Frequency: 0.3 MHz; power intensity: less than 2 W/cm²; duration: less than 30 minutes (each time)). After the first treatment according to the present invention, her liver tumors significantly decreased in size (the imaging study showed that diameters decreased from 10 cm and 7 cm to 7 cm and 4 cm) with no side effect. Several follow up imaging studies showed stable liver metastasis. Her right upper quadrant pain resolved and remained pain free over 18 months till last follow up.

### EXAMPLE 10

A 50 years old female was diagnosed with terminal metastatic ovarian cancer with colostomy and complained of abdominal pain.

The patient was treated twice (one week apart) according to the present invention combined with 30 mg Bleomycin each time (Frequency: 0.3 MHz; power intensity: less than 2 W/cm²; duration: less than 30 minutes (each time)). After the first treatment according to the present invention, her abdominal pain resolved with no side effect. She remained pain free for over 8 months till her death due to sepsis.

As shown in Examples 1 to 10, the sound wave treatment method of the present invention alleviates the symptoms, decreases the tumor size and reduces the tumor markers. Hence, the sound wave treatment method of the present invention provides significant efficacy of tumor treatment. Moreover, the present invention is beneficial, for decreasing the dosage of the anti-tumor drug and reducing the side effects. In addition, for the late-stage caner, the treatment according to the present invention leads to significant efficacy in the treatment. Furthermore, the present invention also is efficient for treating the subject having the drug-resistance.

The present invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

1. A sound wave treatment platform, comprising:
a sound wave emitting module, including a plurality of sound wave emitting devices for emitting non-focused sound waves;
a control module connected to the sound wave emitting module for controlling the sound wave emitting module; and
a supporting component for receiving and supporting the sound wave emitting module.

2. The sound wave treatment platform of claim 1, wherein the sound wave emitting devices are used at a frequency no more than 0.5 MHz.

3. The sound wave treatment platform of claim 1, wherein each of the sound wave emitting devices is independently and operated by the control module.

4. The sound wave treatment platform of claim 1, wherein the sound wave emitting devices are low-frequency ultrasonic transducers, and the non-focused sound waves are non-focused ultrasonic waves.

5. The sound wave treatment platform of claim 1, wherein the sound wave emitting devices are tweeters.

6. The sound wave treatment platform of claim 1, wherein the non-focused sound waves are continuous waves or pulsed waves.

7. The sound wave treatment platform of claim 1, further comprising a connecting component for connecting the supporting component and the control module.

8. The sound wave treatment platform of claim 1, being used for a systemic treatment.

9. The sound wave treatment platform of claim 1, being used for a local treatment.

10. The sound wave treatment platform of claim 1, wherein the supporting component is a bed.

11. The sound wave treatment platform of claim 1, wherein the supporting component has an elastic element for supporting the sound wave emitting devices of the sound wave emitting module.

12. The sound wave treatment platform of claim 1, being used with a drug treatment.

13. The sound wave treatment platform of claim 12, wherein the drug treatment is an anti-tumor drug treatment.

14. The sound wave treatment platform of claim 13, wherein the anti-tumor drug treatment includes a pharmaceutical composition having a tumor-targeting characteristic, a pharmaceutical composition having no tumor-targeting characteristic or a combination thereof.

15. The sound wave treatment platform of claim 13, wherein the anti-tumor drug treatment uses a nano-carrier composition.

16. The sound wave treatment platform of claim 15, wherein the nano-carrier composition includes liposomes, micelles or nano-particles.

17. The sound wave treatment platform of claim 13, wherein the anti-tumor drug treatment is used for treating a cancer.

18. The sound wave treatment platform of claim 1, wherein the sound wave emitting devices fixedly contact a subject to be treated.

19. The sound wave treatment platform of claim 18, wherein the non-focused sound waves are conducted to the subject via a gel pad.

20. The sound wave treatment platform of claim 1, being used with a detecting device.

21. A sound wave treatment method, comprising the step of administering non-focused sound waves to a subject, wherein the non-focused sound waves are produced by a plurality of sound wave emitting devices, and the sound wave emitting devices are used at a frequency no more than 0.5 MHz.

22. The sound wave treatment method of claim 21, wherein the sound wave emitting devices are low-frequency ultrasonic transducers, and the non-focused sound waves are non-focused ultrasonic waves.

23. The sound wave treatment method of claim 21, wherein the sound wave emitting devices are tweeters.

24. The sound wave treatment method of claim 21, wherein the non-focused sound waves are continuous waves or pulsed waves.

25. The sound wave treatment method of claim 21, being used for a systemic treatment.

26. The sound wave treatment method of claim 21, being used for a local treatment.

27. The sound wave treatment method of claim 21, further comprising the step of administering a drug to the subject.

28. The sound wave treatment method of claim 27, wherein the drug is an anti-tumor drug.

29. The sound wave treatment method of claim 28, wherein the anti-tumor drag is a pharmaceutical composition having a tumor-targeting characteristic, a pharmaceutical composition having no tumor-targeting characteristic or a combination thereof.

30. The sound wave treatment method of claim 28, wherein the anti-tumor drug is a nano-carrier composition.

31. The sound wave treatment method of claim 30, wherein the nano-carrier composition includes liposomes, micelles or nano-particles.

32. The sound wave treatment method of claim 28, wherein the anti-tumor drug is an anti-cancer drug.

33. The sound wave treatment method of claim 21, wherein the plurality of sound wave emitting devices are supported by a supporting component.

34. The sound wave treatment method of claim 33, wherein the supporting component is a bed.

35. The sound wave treatment method of claim 21, wherein the sound wave emitting devices fixedly contact the subject.

36. The sound wave treatment method of claim 21, wherein the non-focused sound waves are conducted to the subject via a gel pad, and the gel pad is disposed between the sound wave emitting devices and the subject.

37. The sound wave treatment method of claim 21, wherein each of the sound wave emitting devices is independently operated.

38. The sound wave treatment method of claim 21, wherein the non-focused sound waves are produced by the sound wave treatment platform of claim 1.

39. The sound wave treatment method of claim 21, further comprising the step of using a detecting device for identifying a portion to be treated.

40. The sound wave treatment method of claim 21, further comprising the step of using a detecting device for evaluating an effect of the sound wave treatment method.
